(19) United States
(12) Patent Application Publication
(10) Pub. No.: US 2023/0405009 A1
(43) Pub. Date: Dec. 21, 2023

(54) METHODS FOR TREATING HEMATOLOGIC MALIGNANCIES WITH AMINO-PYRROLOPYRIMIDINONE COMPOUNDS

(71) Applicant: Merck Sharp & Dohme LLC, Rahway, NJ (US)

(72) Inventors: Anson K. Abraham, Furlong, PA (US); Mohammed Z. H. Farooqui, Gwynedd, PA (US); Patricia Marinello, Philadephia, PA (US); Kapil Mayawala, Iselin, NJ (US)

(73) Assignee: Merck Sharp & Dohme LLC, Rahway, NJ (US)

(21) Appl. No.: 18/250,404

(22) PCT Filed: Nov. 2, 2021
(86) PCT No.: PCT/US2021/057710

§ 371 Date: Apr. 25, 2023

Related U.S. Application Data
(60) Provisional application No. 63/109,940, filed on Nov. 5, 2020.

(51) Int. Cl. A61K 31/519; A61P 35/00

(52) U.S. Cl. CPC A61K 31/519; A61P 35/00

(57) ABSTRACT

The application relates to methods of treating a BTK mediated disorder, such as a hematological malignancy, comprising administering to a subject in need thereof a compound of Formula (I) or a pharmaceutically acceptable salt thereof, in an amount from about 80 mg to about 160 mg per day.

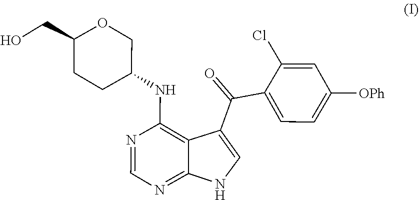 [SMILES: O=C(c1ccc(Oc2ccccc2)cc1Cl)c1c[nH]c2ncnc(N[C@@H]3CC[C@@H](CO)OC3)c12]

### FIELD OF THE INVENTION

The present application is directed to methods of treatment of diseases or disorders associated with Bruton's Tyrosine Kinase (BTK), including hematologic malignancies, immune disorders, cancer, cardiovascular diseases, viral infections, inflammation, metabolism/endocrine function disorders, and neurological disorders, with inhibitors of BTK.

### BACKGROUND OF THE INVENTION

BTK is a member of the Tec family of tyrosine kinases and plays an important role in the regulation of early B-cell development and mature B-cell activation and survival. Functioning downstream of multiple receptors, such as growth factors, B-cell antigen, chemokine, and innate immune receptors, BTK initiates a number of cellular processes including cell proliferation, survival, differentiation, motility, angiogenesis, cytokine production, and antigen presentation.

BTK-deficient mouse models have shown the role BTK plays in allergic disorders and/or autoimmune disease and/or inflammatory disease. For instance, BTK deficiency in standard murine preclinical models of systemic lupus erythematosus (SLE) has been shown to result in a marked amelioration of disease progression. Furthermore, BTK-deficient mice can be resistant to developing collagen-induced arthritis and less susceptible to Staphylococcus-induced arthritis. Due to BTK's role in B-cell activation, BTK inhibitors can also be useful as inhibitors of B-cell mediated pathogenic activity (such as autoantibody production). Expression of BTK in osteoclasts, mast cells and monocytes has been shown to be important for the function of these cells. For example, impaired IgE-mediated mast cell activation and reduced TNF-alpha production by activated monocytes has been associated with BTK deficiency in mice and humans. Thus, BTK inhibition can be useful for the treatment of allergic disorders and/or autoimmune and/or inflammatory diseases such as: SLE, rheumatoid arthritis, multiple vasculitides, idiopathic thrombocytopenic purpura (ITP), myasthenia gravis, allergic rhinitis, and asthma.

Moreover, BTK's role in apoptosis demonstrates the utility of inhibition of BTK activity for the treatment of cancers, B-cell lymphoma, leukemia, and other hematological malignancies. Such hematologic malignancies may include Chronic Lymphocytic Leukemia (CLL), Small Lymphocytic Leukemia (SLL), Richter's Transformation (RT), Mantle cell Lymphoma (MCL), Marginal zone Lymphoma (MZL), Follicular Lymphoma (FL), Waldenström's Macroglobulinemia (WM), and the like. In addition, given the role of BTK in osteoclast function, inhibition of BTK activity can be useful for the treatment of bone disorders such as osteoporosis.

CLL is the most common type of leukemia in western countries. CLL is characterized by the clonal proliferation and accumulation of mature, typically CD5-positive B cells within the blood, bone marrow, lymph nodes and spleen. Deletions of the short arm of chromosome 17 (17p) are found in 5% to 8% of treatment-naïve patients. These deletions almost always include the prominent tumor suppressor gene TP53, and these patients show marked resistance to chemotherapies. CLL and SLL are different manifestations of the same disease and are managed in the same way. The major difference is a significant number of the abnormal lymphocytes are also found in the bone marrow and blood in CLL, while in SLL the abnormal lymphocytes are predominantly found in the lymph nodes and bone marrow. CLL and SLL are used herein interchangeably but refer to the same population of participants.

MCL is a distinct subtype of Non-Hodgkin's Lymphoma (NHL), accounting for 10% of lymphoma cases. Patients usually present with extensive disease, including widespread lymphadenopathy and bone marrow involvement. MCL is not curable, and relapse is common. Existing chemotherapeutic regimens can cause myelosuppression; therefore, there is an unmet medical need for effective therapies.

MZLs consist of a diverse family of malignancies, which are derived from B cells. MZL originates from memory B lymphocytes harbored in the marginal zone of secondary lymphoid follicles present in the spleen, mucosa-associated lymphoid tissues, and rarely lymph nodes. The development of MZL is associated with chronic BCR activation in most cases, which has implications for BTK inhibition.

FL is the second most common NHL, comprising 17% to 22% of cases. Most patients are initially treated with chemoimmunotherapy or rituximab; however, despite good initial responses, FL is incurable in most patients with poor outcomes including relapse and resistant disease. Data suggests that the tumor microenvironment may contribute to the development and progression of FL, and the interaction of FL cells with immune cells in the tumor may influence the clinical course and response to therapy.

Richter's transformation is a life-threatening complication of CLL and represents a unique biological entity with defined mutational events that are both present in the preceding CLL clone, or are acquired at time of transformation. The development of Richter's transformation is characterized by the onset of B symptoms, rapid growth of lymphadenopathy, extra-nodal disease, significant elevations of Lactate Dehydrogenase (LDH), and associated multiorgan dysfunction from invasive or obstructive processes. Most cases represent transformation to diffuse large B-cell lymphoma (DLBCL) and are historically chemorefractory. Current monotherapy approaches with novel agents have done little to impact upon outcomes.

WM is a rare form of B-cell lymphoma that is characterized by elevated serum levels of IgM and infiltration of the bone marrow and other organs by IgM-producing clonal lymphoplasmacytic cells. Rituximab monotherapy and rituximab in combination with alkylating agents, proteasome inhibitors, nucleoside analogs, and more recently ibrutinib are frequently used in these patients. In WM, tumor-cell survival is influenced through BTK-triggered activation of NF-κ3.

Inhibition of BTK with small molecule inhibitors therefore offers a treatment for hematologic malignancies, immune disorders, cancer, cardiovascular diseases, viral infections, inflammation, metabolism/endocrine function disorders, and neurological disorders. Thus, there remains a considerable need for methods of treating diseases or disorders, such as hematological malignancies, where BTK is involved with inhibitors of BTK.

### SUMMARY OF THE INVENTION

A first aspect of the application relates to a method of treating a BTK mediated disorder, comprising administering to a subject in need thereof the compound of Formula (I):

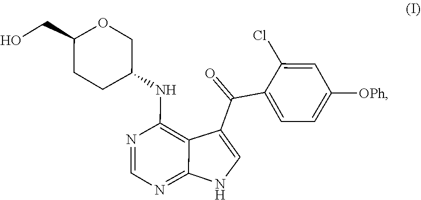 [SMILES: O=C(c1ccc(Oc2ccccc2)cc1Cl)c1c[nH]c2ncnc(N[C@@H]3CC[C@@H](CO)OC3)c12]

or a pharmaceutically acceptable salt thereof, in an amount from about 80 mg to about 160 mg per day. As used herein, the expressions “compound of Formula (I)”, “Compound (I)”, and “Compound A” refer to the same compound and can be used interchangeably. The compound of Formula I is described in U.S. Pat. No. 9,630,968, incorporated by reference herein in its entirety.

Another aspect of the application relates to a method of treating a cell proliferative disorder comprising administering to a subject in need thereof the compound of Formula (I), or a pharmaceutically acceptable salt thereof, in an amount from about 80 mg to about 160 mg per day.

Another aspect of the application relates to a method of treating cancer comprising administering to a subject in need thereof the compound of Formula (I) or a pharmaceutically acceptable salt thereof, in an amount from about 80 mg to about 160 mg per day, wherein the cancer is selected from a hematologic malignancy. Another aspect of the invention relates to a method of treating hematological malignancies wherein the hematological malignancy is selected from Chronic Lymphocytic Leukemia (CLL), Small Lymphocytic Leukemia (SLL), Richter's Transformation (RT), Mantle cell Lymphoma (MCL), Marginal zone Lymphoma (MZL), Follicular Lymphoma (FL), or Waldenström's Macroglobulinemia (WM). As used herein, “hematologic malignancy” and “hematological malignancy” are used interchangeably.

Another aspect of the application relates to a method of modulating (e.g., inhibiting) BTK comprising administering to a subject in need thereof the compound of Formula (I), or a pharmaceutically acceptable salt thereof, in an amount from about 80 mg to about 160 mg per day.

Another aspect of the application relates to the compound of Formula (I) or a pharmaceutically acceptable salt thereof, for use in a method of treating a BTK-mediated disorder, a cell proliferative disorder, or cancer, or hematological malignancy, or of modulating (e.g., inhibiting) BTK, said method comprising administering from about 80 to about 160 mg per day of the compound of Formula (I) or a pharmaceutically acceptable salt thereof to a subject in need thereof.

Another aspect of the application relates to the use of the compound of Formula (I) or a pharmaceutically acceptable salt thereof (e.g., the use of the compound of Formula (I) or a pharmaceutically acceptable salt thereof in an amount from about 80 mg to about 160 mg per day), in the manufacture of a medicament for treating a BTK-mediated disorder, a cell proliferative disorder, or cancer, or hematological malignancy, or for modulating (e.g., inhibiting) BTK. The compound of Formula (I) or a pharmaceutically acceptable salt thereof is for administration or is to be administered in an amount from about 80 mg to about 160 mg per day to a subject in need thereof.

The present application further provides methods of treating a disease or disorder associated with modulation of BTK including, but not limited to, immune disorders, cancer, hematological malignancy, cardiovascular diseases, viral infections, inflammation, metabolism/endocrine function disorders, and neurological disorders comprising administering to a subject suffering from at least one of the diseases or disorders the compound of Formula (I), or a pharmaceutically acceptable salt thereof, in an amount from about 80 mg to about 160 mg per day.

Ultimately the present application provides the medical community with a novel pharmacological strategy for the treatment of diseases and disorders associated with BTK.

In some embodiments, the human patient is administered 80 mg of the compound of Formula I per day. In some embodiments, the human patient is administered 100 mg of the compound of Formula I per day. In some embodiments, the human patient is administered 120 mg of the compound of Formula I per day. In some embodiments, the human patient is administered 140 mg of the compound of Formula I per day. In some embodiments, the human patient is administered 160 mg of the compound of Formula I per day.

### BRIEF DESCRIPTION OF THE DRAWINGS

FIG. 1 is a chart displaying dose escalation and confirmation study details, as well as the number of patients.

FIG. 2 is a chart displaying cohort expansion study details, as well as the number of patients.

### DETAILED DESCRIPTION OF THE INVENTION

The application features methods of treating, preventing or ameliorating a disease or disorder in which BTK plays a role by administering to a subject in need thereof a compound of Formula (I), or a pharmaceutically acceptable salt thereof, in an amount from about 80 mg to about 160 mg per day. The methods of the present application can be used in the treatment of a variety of BTK-mediated diseases and disorders by inhibiting the activity of BTK. Inhibition of BTK provides treatment, prevention, or amelioration of diseases including, but not limited to, immune disorders, cancer, cardiovascular diseases, viral infections, inflammation, metabolism/endocrine function disorders and neurological disorders.

In an aspect of the application, the invention relates to a method of treating a BTK mediated disorder comprising administering to a subject in need thereof the compound of Formula (I):

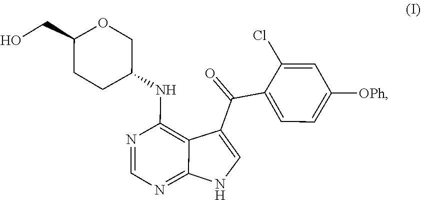 [SMILES: O=C(c1ccc(Oc2ccccc2)cc1Cl)c1c[nH]c2ncnc(N[C@@H]3CC[C@@H](CO)OC3)c12]

or a pharmaceutically acceptable salt thereof, in an amount from about 80 mg to about 160 mg per day.

Another aspect of the application relates to a method of treating a cell proliferative disorder comprising administering to a subject in need thereof the compound of Formula (I), or a pharmaceutically acceptable salt thereof, in an amount from about 80 mg to about 160 mg per day.

Another aspect of the application relates to a method of treating cancer comprising administering to a subject in need thereof the compound of Formula (I), or a pharmaceutically acceptable salt thereof, in an amount from about 80 mg to about 160 mg per day. In an embodiment, the cancer is selected from a hematologic malignancy.

Another aspect of the application relates to a method of modulating (e.g., inhibiting) BTK comprising administering to a subject in need thereof the compound of Formula (I), or a pharmaceutically acceptable salt thereof in an amount from about 80 mg to about 160 mg per day.

Another aspect of the application relates to the compound of Formula (I) or a pharmaceutically acceptable salt thereof, for use in a method of treating a BTK-mediated disorder, a cell proliferative disorder, or cancer, or of modulating (e.g., inhibiting) BTK wherein the method comprises administering a compound of Formula (I), or a pharmaceutically acceptable thereof, in an amount from about 80 mg to about 160 mg per day to a subject in need thereof.

Another aspect of the application relates to the use of the compound of Formula (I) or a pharmaceutically acceptable salt thereof (e.g., the use of the compound of Formula (I) or a pharmaceutically acceptable salt thereof in an amount from about 80 mg to about 160 mg per day), in the manufacture of a medicament for treating a BTK-mediated disorder, a cell proliferative disorder, or cancer, or for modulating (e.g., inhibiting) BTK. The compound of Formula (I) or a pharmaceutically acceptable salt thereof is for administration or is to be administered in an amount from about 80 mg to about 160 mg per day to a subject in need thereof.

For the methods, compound for use, and use of the compound as disclosed in the present application:

In some embodiments, the compound is administered in an amount from about 80 mg to about 160 mg per day. In some embodiments, the compound is administered in an amount of at least 80 mg per day. In some embodiments, the compound is administered in an amount of at least 100 mg per day. In some embodiments, the compound is administered in an amount of at least 120 mg per day.

In some embodiments, the compound is administered in an amount of about 80 mg per day, about 85 mg per day, about 90 mg per day, about 95 mg per day, about 100 mg per day, about 105 mg per day, about 110 mg per day, about 115 mg per day, about 120 mg per day, about 125 mg per day, about 130 mg per day, about 135 mg per day, 140 mg per day, about 145 mg per day, about 150 mg per day, about 155 mg per day or about 160 mg per day.

In some embodiments, the compound is administered in an amount from about 80 mg to about 120 mg per day. In some embodiments, the compound is administered in an amount from about 80 mg to about 100 mg per day. In some embodiments, the compound is administered in an amount of about 80 mg per day. In some embodiments, the compound is administered in an amount of about 100 mg per day. In some embodiments, the compound is administered in an amount of about 120 mg per day.

For any of the doses disclosed herein, in some embodiments, the compound is administered one, two, three, or four times per day. For example, the compound is administered once per day. For example, the compound is administered twice per day. For example, the compound is administered three times per day. For example, the compound is administered four times per day. For example, the compound is administered once per day in an amount from about 80 mg to about 120 mg per day. For example, the compound is administered twice per day in an amount from about 80 mg to about 120 mg per day. For example, the compound is administered three times per day in an amount from about 80 mg to about 120 mg per day. For example, the compound is administered four times per day in an amount from about 80 mg to about 120 mg per day. For example, the compound is administered once per day in an amount of about 80 mg per day. For example, the compound is administered twice per day in an amount of about 80 mg per day. For example, the compound is administered three times per day in an amount of about 80 mg per day. For example, the compound is administered four times per day in an amount of about 80 mg per day. For example, the compound is administered once per day in an amount of about 100 mg per day. For example, the compound is administered twice per day in an amount of about 100 mg per day. For example, the compound is administered three times per day in an amount of about 100 mg per day. For example, the compound is administered four times per day in an amount of about 100 mg per day.

In some embodiments, the compound is administered every day for at least one, two, three, four, five, six, seven, eight, nine, or ten days. In some embodiments, the compound is administered every day for at least one, two, three, four, five, six, seven, eight, nine, or ten weeks. In some embodiments, the compound is administered for at least one day per week, at least two days per week, at least three days per week, at least four days per week, at least five days per week, or at least six days per week.

In some embodiments, the compound is administered for one day per week, two days per week, three days per week, four days per week, five days per week, six days per week, or seven days per week. In some embodiments, the compound is administered on consecutive days. In some embodiments, the compound is administered on alternate days. In some embodiments, the compound is administered on consecutive days, followed by no administration of the compound, followed by administration of the compound, per week.

In some embodiments, the compound is administered for a period of at least one week, two weeks, three weeks, four weeks, five weeks, six weeks, seven weeks, eight weeks, nine weeks, or ten weeks.

In some embodiments, the compound is administered for a period of at least three months, four months, five months, six months, seven months, eight months, nine months, ten months, eleven months, twelve months, eighteen months, twenty-four months, three years, four years, or five years.

The compound of Formula I, or a pharmaceutically acceptable salt thereof, may be administered by doses administered, e.g., daily, 1-7 times per week, weekly, bi-weekly, tri-weekly, every four weeks, every five weeks, every 6 weeks, monthly, bimonthly, quarterly, semiannually, annually, etc. Doses may be administered, e.g., intravenously, subcutaneously, topically, orally, nasally, rectally, intramuscular, intracerebrally, intraspinally, or by inhalation. In certain embodiments, the doses are administered intravenously. In certain embodiments, the doses are administered subcutaneously. In certain embodiments, the doses are administered orally. A total dose for a treatment interval is generally at least 0.05 μg/kg body weight, more generally at least 0.2 μg/kg, 0.5 μg/kg, 1 μg/kg, 10 μg/kg, 100 μg/kg, 0.25 mg/kg, 1.0 mg/kg, 2.0 mg/kg, 5.0 mg/ml, 10 mg/kg, 25 mg/kg, 50 mg/kg or more.

In some embodiments, the compound is administered for a period until the patient shows no symptoms of the diseases or disorders. In some embodiments, the compound is administered for a period until the patient is cured of the diseases or disorders. In some embodiments, the compound is administered for a period until the patient shows resistance to the compound. In some embodiments, the compound is administered for a period until the patient shows side effects that would require discontinuation of the therapy with the compound.

In some embodiments, the compound is administered orally or for oral administration.

In some embodiments, the subject is fasting for at least 1 hour, 2 hours, 3 hours, 4 hours, 6 hours, 8 hours, 12 hours before the compound is administered. In some embodiments, the subject needs to fast for at least 1 hour, 2 hours, 3 hours, 4 hours, 6 hours, 8 hours, 12 hours after the compound is administered.

In some embodiments, the BTK-mediated disorder is selected from immune disorders, cancer, cardiovascular diseases, viral infections, inflammation, metabolism/endocrine function disorders and neurological disorders.

In some embodiments, the cancer is selected from breast cancer, ovary cancer, cervix cancer, prostate cancer, testis cancer, genitourinary tract cancer, esophagus cancer, larynx cancer, glioblastoma, neuroblastoma, stomach cancer, skin cancer, keratoacanthoma, lung cancer, epidermoid carcinoma, large cell carcinoma, non-small cell lung carcinoma (NSCLC), small cell carcinoma, lung adenocarcinoma, bone cancer, colon cancer, adenoma, pancreas cancer, adenocarcinoma, follicular carcinoma, undifferentiated carcinoma, papillary carcinoma, seminoma, melanoma, sarcoma, myeloid disorders, lymphoma, cancer of hairy cells, buccal cavity, naso-pharyngeal, pharynx, lip, tongue, mouth, small intestine, colon-rectum, large intestine, rectum, brain or central nervous system, bronchus, thyroid, liver, intrahepatic bile duct, gastric, endometrial, kidney, renal pelvis, urinary bladder, uterine corpus, or uterine, glioma/glioblastoma, Hodgkin's leukemia, acute myelogenous leukemia, chronic myelogenous leukemia, lymphocytic leukemia, chronic lymphoid leukemia (CLL), Richter's Transformation (RT), small lymphocytic lymphoma (SLL), diffuse large B-cell lymphoma (DLBCL), follicular lymphoma (FL), mantle cell lymphoma (MCL), primary central nervous system (CNS) lymphoma, secondary central nervous system (CNS) lymphoma, marginal zone lymphoma (MZL), Waldenström's macroglobulinemia (WM), acute myeloid leukemia (AML), multiple myeloma (MM), pediatric sarcoma and pediatric brain tumors, myeloid leukemia, oral cavity and pharynx, non-Hodgkin lymphoma, B-cell non-Hodgkin lymphoma, melanoma, and villous colon adenoma.

In some embodiments, the cancer is selected from chronic lymphocytic leukemia (CLL), Richter's Transformation, small lymphocytic lymphoma (SLL), diffuse large B-cell lymphoma (DLBCL), follicular lymphoma (FL), mantle cell lymphoma (MCL), primary central nervous system (CNS) lymphoma, secondary central nervous system (CNS) lymphoma, marginal zone lymphoma (MZL), Waldenström's macroglobulinemia (WM), acute myeloid leukemia (AML), multiple myeloma (MM), pediatric sarcoma, and pediatric brain tumors. In some embodiments, the cancer is a B-cell lymphoid malignancy. In some embodiments, the cancer is selected from CLL, Richter's Transformation, FL, and DLBCL.

In some embodiments, the cancer is selected a hematological malignancy. In some embodiments, the hematological malignancy is selected from Chronic Lymphocytic Leukemia (CLL), Small Lymphocytic Leukemia (SLL), Richter's Transformation (RT), Mantle cell Lymphoma (MCL), Marginal zone Lymphoma (MZL), Follicular Lymphoma (FL), Waldenström's Macroglobulinemia (WM). In some embodiments, the hematological malignancy is CLL. In some embodiments, the hematological malignancy is SLL. In some embodiments, the hematological malignancy is RT. In some embodiments, the hematological malignancy is MCL. In some embodiments, the hematological malignancy is MZL. In some embodiments, the hematological malignancy is FL. In some embodiments, the hematological malignancy is WM.

In some embodiments, the cancer has a mutant BTK. In some embodiments, the cancer has a BTK C481 mutation. In some embodiments, the cancer has a BTK C481S mutation.

In some embodiments, the cancer is relapsed or refractory to at least one prior therapy. In some embodiments, the cancer is relapsed or refractory to at least one therapy with a BTK inhibitor. For example, in some embodiments, the cancer is ibrutinib resistant.

In one embodiment, the invention is directed to a pharmaceutically acceptable salt of the compound of Formula (I). In another embodiment, the invention is directed to a hydrate of the the compound of Formula (I). In yet another embodiment, the invention is directed to a solvate of the compound of Formula (I).

The details of the application are set forth in the accompanying description below. Although methods and materials similar or equivalent to those described herein can be used in the practice or testing of the present application, illustrative methods and materials are now described. Other features, objects, and advantages of the application will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this application belongs. All patents and publications cited in this specification are incorporated herein by reference in their entireties.

### Definitions

The articles “a” and “an” are used in this application to refer to one or more than one (i.e., at least one) of the grammatical object of the article. By way of example, “an element” means one element or more than one element.

The application also includes pharmaceutical compositions comprising an effective amount of the compound of Formula (I), as disclosed herein, and a pharmaceutically acceptable carrier.

The term “carrier”, as used in this application, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The compound of Formula (I) may form salts which are also within the scope of this application. The present invention is directed to a compound of Formula (I) and pharmaceutically acceptable salts thereof.

The compound of the present application and pharmaceutically acceptable salts, tautomers, prodrugs, and polymorphs of the compound of Formula I thereof can exist in a solvated form with other solvent molecules or in an unsolvated form.

“Solvate” means solvent addition forms that contain either stoichiometric or non stoichiometric amounts of solvent. Some compounds or salts have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

All stereoisomers (for example, geometric isomers, optical isomers and the like) of the present compound (including those of the salts, solvates, esters and prodrugs of the compound as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this application, as are positional isomers (such as, for example, 4-pyridyl and 3-pyridyl). For example, if a compound of Formula (I) incorporates a double bond or a fused ring, both the cis- and trans-forms, as well as mixtures, are embraced within the scope of the application. Individual stereoisomers of the compound of the application may, for example, be substantially free of other isomers, or may be admixed, for example, as racemates or with all other, or other selected, stereoisomers. The chiral centers of the present application can have the S or R configuration as defined by the IUPAC 1974 Recommendations. The use of the terms “salt”, “solvate”, “ester,” “prodrug” and the like, is intended to equally apply to the salt, solvate, ester and prodrug of enantiomers, stereoisomers, rotamers, tautomers, positional isomers, racemates or prodrugs of the inventive compound.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compound of Formula (I) may have one or more asymmetric carbon atom and may occur as racemates, racemic mixtures or as individual enantiomers or diastereomers.

In the present specification, the structural formula of the compound represents a certain isomer for convenience in some cases, but the present application includes all isomers, such as geometrical isomers, optical isomers based on an asymmetrical carbon, stereoisomers, tautomers, and the like.

“Isomerism” means compounds that have identical molecular formulae but differ in the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images of each other are termed “enantiomers” or sometimes optical isomers. A mixture containing equal amounts of individual enantiomeric forms of opposite chirality is termed a “racemic mixture”.

The compound of Formula I may contain asymmetric or chiral centers, and, therefore, exist in different stereoisomeric forms. It is intended that all stereoisomeric forms of the compound of the application as well as mixtures thereof, including racemic mixtures, form part of the present application. In addition, the present application embraces all geometric and positional isomers. For example, if a compound of the application incorporates a double bond or a fused ring, both the cis- and trans-forms, as well as mixtures, are embraced within the scope of the application. Each compound herein disclosed includes all the enantiomers that conform to the general structure of the compound. The compound may be in a racemic or enantiomerically pure form, or any other form in terms of stereochemistry. The assay results may reflect the data collected for the racemic form, the enantiomerically pure form, or any other form in terms of stereochemistry.

A carbon atom bonded to four non-identical substituents is termed a “chiral center”. “Chiral isomer” means a compound with at least one chiral center. Compounds with more than one chiral center may exist either as an individual diastereomer or as a mixture of diastereomers, termed “diastereomeric mixture”. When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

In another embodiment of the application, the compound of Formula (I) is an enantiomer. In some embodiments the compound is the (S)-enantiomer. In other embodiments the compound is the (R)-enantiomer. In yet other embodiments, the compound of Formula (I) may be (+) or (−) enantiomers. The compound may contain more than one stereocenter.

In another embodiment of the application, the compound of Formula (I) are diastereomers. In some embodiments, the compound are the syn diastereomer. In other embodiments, the compound are the anti diastereomer.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Enantiomers can also be separated by use of a chiral HPLC column.

It is also possible that the compound of the application may exist in different tautomeric forms, and all such forms are embraced within the scope of the application. Also, for example, all keto-enol and imine-enamine forms of the compound are included in the application.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solid form, usually one tautomer predominates. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), amine-enamine and enamine-imine. (Pyrrolopyrimidinyl)methanone-(Pyrrolopyrimidinyl)methanol tautomeric pairs are included in the present application:

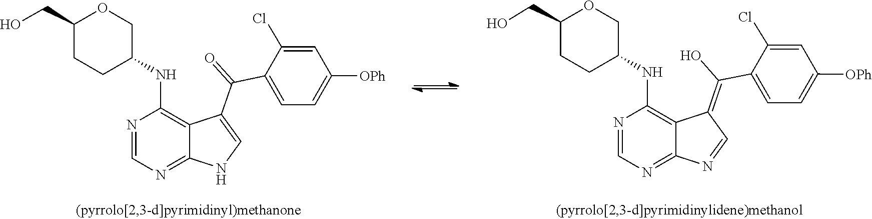 [SMILES: O=C(c1ccc(Oc2ccccc2)cc1Cl)c1c[nH]c2ncnc(N[C@@H]3CC[C@@H](CO)OC3)c12 ; OC[C@@H]1CC[C@@H](Nc2ncnc3c2/C(=C(\O)c2ccc(Oc4ccccc4)cc2Cl)C=N3)CO1]

The present application relates to the compound of Formula (I) or pharmaceutically acceptable salts thereof, capable of inhibiting BTK, which are useful for the treatment of diseases and disorders associated with modulation of BTK. The application further relates to the compound of Formula (I), or pharmaceutically acceptable salts thereof, which are useful for inhibiting BTK. In some embodiments, the BTK is wild-type BTK. In other embodiments, the BTK is a mutant BTK.

The present application relates to a compound of Formula (I), wherein the compound inhibits kinase activity of a mutant BTK, such as a drug-resistant mutant BTK harboring a drug-resistance mutation (e.g., C481S mutation). In some embodiments, the patient or subject does not respond to a BTK inhibitor or relapse after the treatment of a BTK inhibitor, due to a mutation of BTK (e.g., a C481S mutation) that prevents target inhibition. In one embodiment, the BTK mutation is a C481S mutation.

As used herein, “relapsed” and “refractory” are defined as follows: Relapsed disease is disease progression following at least one line or prior therapy and Refractory disease is failure to achieve a complete response (CR) or partial response (PR) with at least one line of prior therapy. In some embodiments, the relapsed disease, such as a relapsed hematological malignancy, is disease progression following the most recent therapy. In some embodiments, refractory disease, such as a relapsed hematological malignancy, is failure to achieve CR or PR with the most recent therapy.

The compound of the present application can be converted to N-oxides by treatment with an oxidizing agent (e.g., 3-chloroperoxybenzoic acid (m-CPBA) and/or hydrogen peroxides) to afford other compounds of the present application. Thus, all shown and claimed nitrogen-containing compounds are considered, when allowed by valency and structure, to include both the compound as shown and its N-oxide derivative (which can be designated as N→O or N⁺—O⁻). Furthermore, in other instances, the nitrogens in the compound of the present application can be converted to N-hydroxy or N-alkoxy compounds. For example, N-hydroxy compounds can be prepared by oxidation of the parent amine by an oxidizing agent such as m-CPBA. All shown and claimed nitrogen-containing compounds are also considered, when allowed by valency and structure, to cover both the compounds as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

The term “prodrug,” as used in this application, means a compound which is convertible in vivo by metabolic means (e.g., by hydrolysis) to the compound of Formula I.

Since prodrugs are known to enhance numerous desirable qualities of pharmaceuticals (e.g., solubility, bioavailability, manufacturing, etc.) the compound of Formula (I), or pharmaceutically acceptable salts thereof can be delivered in prodrug form. Thus, the present application is intended to cover prodrugs of the compound of Formula (I), or a pharmaceutically acceptable salt thereof, methods of delivering the same and compositions containing the same. “Prodrugs” are intended to include any covalently bonded carriers that release an active parent drug of the present application in vivo when such prodrug is administered to a mammalian subject. Prodrugs are prepared by modifying functional groups present in the compound in such a way that the modifications are cleaved, either in routine manipulation or in vivo, to the parent compound. Prodrugs include compounds of the application wherein a hydroxyl or amino, group is bonded to any group that, when the prodrug of the present application is administered to a mammalian subject, it cleaves to form a free hydroxyl or free amino group, respectively. Examples of prodrugs include, but are not limited to, acetate, formate, and benzoate derivatives of alcohol and amine functional groups in the compound of Formula I or a pharmaceutically acceptable salt thereof.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compound of Formula I can be prepared by crystallization under different conditions.

The application also comprehends isotopically-labeled compounds, which are identical to those recited in the each of the formulae described herein, but for the fact that one or more atoms are replaced by an atom having an atomic mass or mass number different from the atomic mass or mass number most commonly found in nature. Examples of isotopes that can be incorporated into the compound of the application include isotopes of hydrogen, carbon, nitrogen, fluorine, such as ³H, ¹¹C, ¹⁴C, ²H and ¹⁸F.

The compound of Formula (I), or pharmaceutically acceptable salts thereof, that contains the aforementioned isotopes and/or other isotopes of other atoms are within the scope of the present application. Isotopically-labeled compounds of the present application, for example those into which radioactive isotopes such as ³H, ¹⁴C are incorporated, are useful in drug and/or substrate tissue distribution assays. Tritiated, i.e., ³H, and carbon-14, i.e., ¹⁴C isotopes are useful for their ease of preparation and detectability. ¹¹C and ¹⁸F isotopes are useful in PET (positron emission tomography). PET is useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances, isotopically labeled compounds of Formula (I), or pharmaceutically acceptable salts thereof, can generally be prepared by carrying out the procedures disclosed in the Schemes and/or in the Examples described herein, by substituting a readily available isotopically labeled reagent for a non-isotopically labeled reagent. In one embodiment, the compound of Formula (I) or pharmaceutically acceptable salts thereof, are not isotopically labelled.

The compound of the present application can be prepared in a number of ways well known to those skilled in the art of organic synthesis, such as the method described in U.S. Pat. No. 9,630,968.

The term “administer”, “administering”, or “administration” as used in this application refers to either directly administering a compound of Formula I or a pharmaceutically acceptable salt thereof or a composition containing the compound of Formula I to a subject.

A “patient” or “subject” is a mammal, e.g., a human, mouse, rat, guinea pig, dog, cat, horse, cow, pig, or non-human primate, such as a monkey, chimpanzee, baboon or rhesus.

An “effective amount” or “therapeutically effective amount” when used in connection with a compound or pharmaceutical composition is an amount effective for treating or preventing a disease in a subject as described herein.

The term “treating” with regard to a subject, refers to improving at least one symptom of the subject's disorder. Treating includes curing, improving, or at least partially ameliorating the disorder.

The compound of the present application, or a pharmaceutically acceptable salt thereof, can also be used to prevent a disease, condition or disorder. As used herein, “preventing” or “prevent” describes reducing or eliminating the onset of the symptoms or complications of the disease, condition or disorder.

The term “disorder” is used in this application to mean, and is used interchangeably with, the terms disease, condition, or illness, unless otherwise indicated.

As used herein, the term “BTK-mediated” diseases or disorders means any disease or other deleterious condition in which BTK, or a mutant thereof, is known to play a role. Accordingly, another embodiment of the present application relates to treating or lessening the severity of one or more diseases in which BTK, or a mutant thereof, is known to play a role. Specifically, the present application relates to a method of treating or lessening the severity of a disease or condition selected from a proliferative disorder or an autoimmune disorder, wherein said method comprises administering to a patient in need thereof a compound of Formula (I), or pharmaceutically acceptable salts thereof, or a composition according to the present application.

As used herein, the term “cell proliferative disorder” refers to conditions in which unregulated or abnormal growth, or both, of cells can lead to the development of an unwanted condition or disease, which may or may not be cancerous. Exemplary cell proliferative disorders of the application encompass a variety of conditions wherein cell division is deregulated. Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring or juxtaposed cells within the same tissue. A cell proliferative disorder includes a precancer or a precancerous condition. A cell proliferative disorder includes cancer. Preferably, the methods provided herein are used to treat or alleviate a symptom of cancer. The term “cancer” includes solid tumors, as well as, hematologic tumors and/or malignancies. A “precancer cell” or “precancerous cell” is a cell manifesting a cell proliferative disorder that is a precancer or a precancerous condition. A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

Exemplary non-cancerous conditions or disorders include, but are not limited to, rheumatoid arthritis; inflammation; autoimmune disease; chronic Graft Versus Host Disease (cGVHD), lymphoproliferative conditions; acromegaly; rheumatoid spondylitis; osteoarthritis; gout, other arthritic conditions; sepsis; septic shock; endotoxic shock; gram-negative sepsis; toxic shock syndrome; asthma; adult respiratory distress syndrome; chronic obstructive pulmonary disease; chronic pulmonary inflammation; inflammatory bowel disease; Crohn's disease; psoriasis; eczema; ulcerative colitis; pancreatic fibrosis; hepatic fibrosis; acute and chronic renal disease; irritable bowel syndrome; pyresis; restenosis; cerebral malaria; stroke and ischemic injury; neural trauma; Alzheimer's disease; Huntington's disease; Parkinson's disease; acute and chronic pain; allergic rhinitis; allergic conjunctivitis; chronic heart failure; acute coronary syndrome; cachexia; malaria; leprosy; leishmaniasis; Lyme disease; Reiter's syndrome; acute synovitis; muscle degeneration, bursitis; tendonitis; tenosynovitis; herniated, ruptures, or prolapsed intervertebral disk syndrome; osteopetrosis; thrombosis; restenosis; silicosis; pulmonary sarcosis; bone resorption diseases, such as osteoporosis; graft-versus-host reaction; Multiple Sclerosis; lupus; fibromyalgia; AIDS and other viral diseases such as Herpes Zoster, Herpes Simplex I or II, influenza virus and cytomegalovirus; and diabetes mellitus.

Exemplary cancers include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, B-cell non-Hodgkin lymphoma, non-Hodgkin lymphoma, primary central nervous system lymphoma, Waldenström's macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, chronic lymphocytic leukemia (CLL), Richter's Transformation, small lymphocytic lymphoma (SLL), diffuse large B-cell lymphoma (DLBCL), follicular lymphoma (FL), mantle cell lymphoma (MCL), primary central nervous system (CNS) lymphoma, secondary central nervous system (CNS) lymphoma, marginal zone lymphoma (MZL), Waldenström's macroglobulinemia (WM), acute myeloid leukemia (AML), multiple myeloma (MM), pediatric sarcoma, pediatric brain tumors, and Wilm's Tumor.

### Methods of Using the Compound

The application relates to a method of treating, preventing, inhibiting, or eliminating a disease or disorder associated with modulation of BTK (e.g., inhibition of BTK), such as the disease or disorder disclosed herein. The method comprises administering to a patient in need of a treatment for diseases or disorders associated with modulation of BTK an amount of the compound of Formula (I), as disclosed herein, or a pharmaceutically acceptable salt thereof or a pharmaceutical composition of the compound of Formula (I). In one embodiment, the BTK-mediated disorder is selected from immune disorders, cancer, cardiovascular diseases, viral infections, inflammation, metabolism/endocrine function disorders and neurological disorders. In some embodiments, the method further comprises administering an additional therapeutic agent selected from an anti-inflammatory agent, an immunomodulatory agent, chemotherapeutic agent, a neurotropic factor, an agent for treating cardiovascular disease, an agent for treating liver disease, an anti-viral agent, an agent for treating blood disorders, an agent for treating diabetes, and an agent for treating immunodeficiency disorders. In some embodiments, the BTK is wild-type BTK. In other embodiments, the BTK is mutant BTK (e.g., BTK C481S mutant).

Another aspect of the application relates to a method of treating, preventing, inhibiting, or eliminating a cell proliferative disorder, such as the cell proliferative disorder disclosed herein, the method comprising administering to a patient in need thereof an amount of the compound of Formula (I), as disclosed herein, or a pharmaceutically acceptable salt thereof or a pharmaceutical composition of the compound of Formula (I). In one embodiment, the cell proliferative disorder is a cancer, such as the cancer disclosed herein. In some embodiments, the method further comprises administering an additional therapeutic agent selected from an anti-inflammatory agent, an immunomodulatory agent, chemotherapeutic agent, a neurotropic factor, an agent for treating cardiovascular disease, an agent for treating liver disease, an anti-viral agent, an agent for treating blood disorders, an agent for treating diabetes, and an agent for treating immunodeficiency disorders.

Another aspect of the application relates to a method of modulating BTK, the method comprising administering to a patient in need thereof an amount of the compound of Formula (I), as disclosed herein, or a pharmaceutically acceptable salt thereof or a pharmaceutical composition of the compound of Formula (I). In one embodiment, modulating BTK is inhibiting BTK. In some embodiments, the BTK is wild-type BTK. In other embodiments, the BTK is mutant BTK (e.g., BTK C481S mutant).

Another aspect of the application relates to the compound of Formula (I), or a pharmaceutically acceptable salt thereof, for use in a method of treating a BTK-mediated disorder, such as the disorder disclosed herein. In one embodiment, the compound of Formula (I) is for use at the amount disclosed herein. In one embodiment, the disease or disorder is selected from immune disorders, cancer, cardiovascular diseases, viral infections, inflammation, metabolism/endocrine function disorders and neurological disorders. In some embodiments, the method further comprises administering an additional therapeutic agent selected from an anti-inflammatory agent, an immunomodulatory agent, chemotherapeutic agent, a neurotropic factor, an agent for treating cardiovascular disease, an agent for treating liver disease, an anti-viral agent, an agent for treating blood disorders, an agent for treating diabetes, and an agent for treating immunodeficiency disorders. In some embodiments, the BTK is wild-type BTK. In other embodiments, the BTK is mutant BTK (e.g., BTK C481S mutant).

Another aspect of the application relates to the compound of Formula (I), or a pharmaceutically acceptable salt thereof, for use in a method of treating, preventing, inhibiting, or eliminating a cell proliferative disorder, such as the cell proliferative disorder disclosed herein. In one embodiment, the cell proliferative disorder is a cancer, such as the cancer disclosed herein. In one embodiment, the compound of Formula (I) is for use at the amount disclosed herein.

Another aspect of the application relates to the compound of Formula (I), or a pharmaceutically acceptable salt thereof, for use in modulating BTK. In one embodiment, modulating BTK is inhibiting BTK. In some embodiments, the BTK is wild-type BTK. In other embodiments, the BTK is mutant BTK (e.g., BTK C481S mutant). In one embodiment, the compound of Formula (I) is for use at the amount disclosed herein.

Another aspect of the application relates to the use of the compound of Formula (I), or a pharmaceutically acceptable salt thereof, in the manufacture of a medicament for treating a BTK-mediated disease or disorder, such as the disease or disorder disclosed herein. In one embodiment, the use comprises use of the compound of Formula (I) at the amount disclosed herein. In one embodiment, the disease or disorder is selected from immune disorders, cancer, cardiovascular diseases, viral infections, inflammation, metabolism/endocrine function disorders and neurological disorders. In some embodiments, the treatment further comprises administering an additional therapeutic agent selected from an anti-inflammatory agent, an immunomodulatory agent, chemotherapeutic agent, a neurotropic factor, an agent for treating cardiovascular disease, an agent for treating liver disease, an anti-viral agent, an agent for treating blood disorders, an agent for treating diabetes, and an agent for treating immunodeficiency disorders. In some embodiments, the BTK is wild-type BTK. In other embodiments, the BTK is mutant BTK (e.g., BTK C481S mutant).

Another aspect of the application relates to the use of the compound of Formula (I), or a pharmaceutically acceptable salt thereof, in the manufacture of a medicament for treating, preventing, inhibiting, or eliminating a cell proliferative disorder, such as the cell proliferative disorder disclosed herein. In one embodiment, the cell proliferative disorder is a cancer, such as the cancer disclosed herein. In one embodiment, the use comprises use of the compound of Formula (I) at the amount disclosed herein.

Another aspect of the application relates to the use of the compound of Formula (I), or a pharmaceutically acceptable salt thereof, in the manufacture of a medicament for modulating BTK. In one embodiment, modulating BTK is inhibiting BTK. In some embodiments, the BTK is wild-type BTK. In other embodiments, the BTK is mutant BTK (e.g., BTK C481S mutant). In one embodiment, the use comprises use of the compound of Formula (I) at the amount disclosed herein.

In some embodiments of the methods and uses described herein, the cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, pancreatic, myeloid disorders, lymphoma, hairy cells, buccal cavity, naso-pharyngeal, pharynx, lip, tongue, mouth, small intestine, colon-rectum, large intestine, rectum, brain and central nervous system, Hodgkin's leukemia, bronchus, thyroid, liver and intrahepatic bile duct, hepatocellular, gastric, glioma/glioblastoma, endometrial, melanoma, kidney and renal pelvis, urinary bladder, uterine corpus, uterine cervix, multiple myeloma, acute myelogenous leukemia, chronic myelogenous leukemia, lymphocytic leukemia, chronic lymphoid leukemia (CLL), myeloid leukemia, oral cavity and pharynx, B-cell non-Hodgkin lymphoma, non-Hodgkin lymphoma, melanoma, and villous colon adenoma.

In some embodiments of the methods and uses described herein, the cancer is selected from chronic lymphocytic leukemia (CLL), Richter's Transformation, small lymphocytic lymphoma (SLL), diffuse large B-cell lymphoma (DLBCL), follicular lymphoma (FL), mantle cell lymphoma (MCL), primary central nervous system (CNS) lymphoma, secondary central nervous system (CNS) lymphoma, marginal zone lymphoma (MZL), Waldenström's macroglobulinemia (WM), acute myeloid leukemia (AML), multiple myeloma (MM), pediatric sarcoma and pediatric brain tumors.

In any of the embodiments of the application, the cancer can be any cancer in any organ, for example, a cancer is selected from the group consisting of glioma, thyroid carcinoma, breast carcinoma, small-cell lung carcinoma, non-small-cell carcinoma, gastric carcinoma, colon carcinoma, gastrointestinal stromal carcinoma, pancreatic carcinoma, bile duct carcinoma, CNS carcinoma, ovarian carcinoma, endometrial carcinoma, prostate carcinoma, renal carcinoma, anaplastic large-cell lymphoma, leukemia, multiple myeloma, mesothelioma, and melanoma, and combinations thereof.

In some embodiments of the methods and uses described herein, the disease or disorder is an immune disorder. In one embodiment, the immune disorder is rheumatoid arthritis. In some embodiments of the methods and uses described herein, the disease or disorder is systemic and local inflammation, arthritis, inflammation related to immune suppression, organ transplant rejection, allergies, ulcerative colitis, Crohn's disease, dermatitis, asthma, systemic lupus erythematosus, Sjogren's Syndrome, multiple sclerosis, scleroderma/systemic sclerosis, idiopathic thrombocytopenic purpura (ITP), anti-neutrophil cytoplasmic antibodies (ANCA) vasculitis, chronic obstructive pulmonary disease (COPD), psoriasis.

In one embodiment, methods of treating a disease or disorder associated with modulation of BTK including, immune disorders, cancer, cardiovascular diseases, viral infections, inflammation, metabolism/endocrine function disorders and neurological disorders, comprise administering to a patient suffering from at least one of the diseases or disorder the compound of Formula (I).

The compound of Formula I can be administered in effective amounts to treat or prevent a disorder and/or prevent the development thereof in subjects, such as the amount disclosed herein.

The compound of Formula I can be administered in therapeutically effective amounts in a combinational therapy with one or more therapeutic agents (pharmaceutical combinations) or modalities, e.g., non-drug therapies. For example, synergistic effects can occur with other anti-proliferative, anti-cancer, immunomodulatory or anti-inflammatory substances. In some embodiments, the compound of Formula (I) is administered in combination with an additional therapeutic agent selected from an anti-inflammatory agent, an immunomodulatory agent, chemotherapeutic agent, a neurotropic factor, an agent for treating cardiovascular disease, an agent for treating liver disease, an anti-viral agent, an agent for treating blood disorders, an agent for treating diabetes, and an agent for treating immunodeficiency disorders. Where the compound of the application is administered in conjunction with other therapies, dosages of the co-administered compounds will of course vary depending on the type of co-drug employed, on the specific drug employed, on the condition being treated and so forth.

Combination therapy includes the administration of the subject compound in further combination with other biologically active ingredients (such as, but not limited to, an anti-inflammatory agent, an immunomodulatory agent, chemotherapeutic agent, a neurotropic factor, an agent for treating cardiovascular disease, an agent for treating liver disease, an anti-viral agent, an agent for treating blood disorders, an agent for treating diabetes, and an agent for treating immunodeficiency disorders) and non-drug therapies (such as, but not limited to, surgery or radiation treatment). For instance, the compound of the application can be used in combination with other pharmaceutically active compounds, preferably compounds that are able to enhance the effect of the compound of the application. The compound of the application can be administered simultaneously (as a single preparation or separate preparation) or sequentially to the other drug therapy or treatment modality. In general, a combination therapy envisions administration of two or more drugs during a single cycle or course of therapy.

### Pharmaceutical Compositions

The present application also discloses pharmaceutical compositions comprising the compound of Formula (I), or a pharmaceutically acceptable salt thereof, in combination with at least one pharmaceutically acceptable excipient or carrier.

A “pharmaceutical composition” is a formulation containing the compound of the present application in a form suitable for administration to a subject. For example, the pharmaceutical composition is in bulk or in unit dosage form. The unit dosage form may be in any of a variety of forms, including, for example, a capsule, an IV bag, a tablet, a single pump on an aerosol inhaler or a vial. The quantity of active ingredient (e.g., a formulation of the compound of Formula I or a pharmaceutically acceptable salt thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical compositions of the application are formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition of the application can be administered to a subject in many of the well-known methods currently used for chemotherapeutic treatment. For example, for treatment of cancers, a compound of the application may be injected directly into tumors, injected into the blood stream or body cavities or taken orally or applied through the skin with patches. The dose chosen should be sufficient to constitute effective treatment but not as high as to cause unacceptable side effects. The state of the disease condition (e.g., cancer, precancer, and the like) and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. For example, the disease or disorder is selected from immune disorders, cancer, cardiovascular diseases, viral infections, inflammation, metabolism/endocrine function disorders and neurological disorders.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy.

The pharmaceutical compositions containing active compound (i.e., the compound of Formula (I)) of the present application may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compound into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compound is delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compound is formulated into ointments, salves, gels, or creams as generally known in the art.

The active compound can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the application are dictated by and directly dependent on the unique characteristics of the active compound and the particular therapeutic effect to be achieved.

In therapeutic applications, the dosages of the pharmaceutical compositions used in accordance with the application vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

The pharmaceutical compositions can be included in a container, pack, or dispenser together with instructions for administration.

As used herein, “pharmaceutically acceptable salts” refer to derivatives of the compound of the present application wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Additional representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present application also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates) or crystal forms (polymorphs) as defined herein, of the same salt.

The compound of the present application can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., an acetate, propionate or other ester.

The compound of the present application can also be prepared as prodrugs, for example, pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.), the compound of the present application can be delivered in prodrug form. Thus, the present application is intended to cover prodrugs of the presently claimed compound, methods of delivering the same and compositions containing the same. “Prodrugs” are intended to include any covalently bonded carriers that release an active parent drug of the present application in vivo when such prodrug is administered to a subject. Prodrugs in the present application are prepared by modifying functional groups present in the compound in such a way that the modifications are cleaved, either in routine manipulation or in vivo, to the parent compound. Prodrugs include the compound of the present application wherein a hydroxy, amino, sulfhydryl, carboxy or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., ethyl esters, morpholinoethanol esters) of carboxyl functional groups, N-acyl derivatives (e.g., N-acetyl) N-Mannich bases, Schiff bases and enaminones of amino functional groups, oximes, acetals, ketals and enol esters of ketone and aldehyde functional groups in the compound of the application, and the like, See Bundegaard, H., Design of Prodrugs, p 1-92, Elsevier, New York-Oxford (1985).

The compound, or a pharmaceutically acceptable salt thereof, is administered orally, nasally, transdermally, pulmonary, inhalationally, buccally, sublingually, intraperintoneally, subcutaneously, intramuscularly, intravenously, rectally, intrapleurally, intrathecally and parenterally. For example, the compound, or a pharmaceutically acceptable salt thereof, is administered orally. One skilled in the art will recognize the advantages of certain routes of administration.

The dosage regimen utilizing the compound is selected in accordance with a variety of factors including type, species, age, weight, sex and medical condition of the patient; the severity of the condition to be treated; the route of administration; the renal and hepatic function of the patient; and the particular compound or pharmaceutically acceptable salt thereof employed. An ordinarily skilled physician or veterinarian can readily determine and prescribe the effective amount of the drug required to prevent, counter or arrest the progress of the condition.

Techniques for formulation and administration of the compound of Formula I on can be found in Remington: the Science and Practice of Pharmacy, 19 th edition, Mack Publishing Co., Easton, PA (1995). In an embodiment, the compound described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compound or pharmaceutically acceptable salts thereof will be present in such pharmaceutical compositions in amounts sufficient to provide the desired dosage amount in the range described herein.

All percentages and ratios used herein, unless otherwise indicated, are by weight. Other features and advantages of the present application are apparent from the different examples. The provided examples illustrate different components and methodology useful in practicing the present application. The examples do not limit the claimed application. Based on the present application the skilled artisan can identify and employ other components and methodology useful for practicing the present application.

Unless otherwise defined, all technical and scientific terms used herein have the same meaning as commonly understood by one of ordinary skill in the art to which this disclosure relates. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting. Abbreviations listed below may be used in the examples herein.

| ABBREVIATION | DEFINITION |
|---|---|
| AE | Adverse event |
| AR | Adverse reaction |
| AUC | Area under the curve |
| BCL2i | B-cell lymphoma 2 inhibitor |
| BCR | B-cell receptor |
| BTKi | BTK inhibitor |
| CBC | Complete blood cell |
| CI | Confidence interval |
| CNS | Central nervous system |
| CR | Complete response |
| CRi | Complete response with incomplete bone marrow |
| | recovery |
| CTCAE | Common Terminology Criteria for Adverse Events |
| ctDNA | Circulating tumor DNA |
| CYP | Cytochrome P450 |
| D | De-escalate to the next lower dose; |
| DILI | Drug-induced liver injury |
| DLBCL | Diffuse large B-cell lymphoma |
| DLCO | Diffusing capacity for carbon monoxide |
| DLT | Dose-limiting toxicity |
| DLT(e) | Dose-limiting toxicity (evaluable) |
| DOR | Duration of response |
| DU | The current dose is unacceptably toxic |
| E | Escalate to the next higher dose |
| ECG | Electrocardiogram |
| ECI | Event of clinical interest |
| EORTC | European Organization for Research and Treatment |
| | of Cancer |
| EOT | End of treatment |
| ePROs | Electronic patient-reported outcomes |
| eEQ-5D | Electronic EQ-5D |
| EQ-5D | EuroQoL-5D |
| GCP | Good Clinical Practice |
| Hb | hemoglobin |
| IC50 | Half maximal inhibitory concentration |
| IHC | Immunohistochemistry |
| iwCLL | International Workshop on Chronic Lymphocytic |
| | Leukemia |
| IWWM | International Workshop on Waldenström's |
| | macroglobulinemia |
| LDH | Lactate dehydrogenase |
| MR | Minor response |
| MRD | Minimal residual disease |
| MTD | Maximum tolerated dose |
| mTPI | Modified Toxicity Probability Interval |
| n | number |
| NCI | National cancer institute |
| NF-κB | Nuclear factor kappa-light chain-enhancer of |
| | activated B cells |
| NHL | Non-Hodgkin's Lymphoma |
| NIH | National Institute for Health |
| nPR | Nodular partial response |
| OR | Objective response |
| ORR | Objective response rate |
| OS | Overall survival |
| PCR | Polymerase Chain Reaction |
| PD | Disease progression |
| PET | Positron emission tomography |
| PFS | Progression-free survival |
| PI3Ki | Phosphoinositide 3-kinase inhibitor |
| PK | Pharmacokinetic(s) |
| PO | Orally |
| PR | Partial response |
| PRL | Partial response with lymphocytosis |
| PRO | Patient-reported outcome |
| QD | Once daily |
| QLQ | Quality of life questionnaire |
| QOL | Quality of life |
| QT | QT interval |
| QTc | QT interval corrected |
| rrCLL | Relapsed or refractory CLL |
| RP2D | Recommended Phase 2 dose |
| rrDLBCL | Relapsed or refractory DLBCL |
| RV | Residual volume |
| S | Stay at the current dose. |
| SAE | serious adverse event |
| SoA | Schedule of activities |
| VAS | Visual analog scale |
| VGPR | Very good partial response |

Exemplary methods and materials are described herein, although methods and materials similar or equivalent to those described herein can also be used in the practice or testing of the present disclosure. The materials, methods, and examples are illustrative only and not intended to be limiting.

### EXAMPLES

The application is further illustrated by the following examples, which are not to be construed as limiting this application in scope or spirit to the specific procedures herein described. It is to be understood that the examples are provided to illustrate certain embodiments and that no limitation to the scope of the application is intended thereby. It is to be further understood that resort may be had to various other embodiments, modifications, and equivalents thereof which may suggest themselves to those skilled in the art without departing from the spirit of the present application and/or scope of the appended claims.

### Example 1

### Phase 2, Open-Label, Nonrandomized Dose-Escalation and Cohort Expansion Study

Despite major therapeutic advances in B-cell malignancies, cases of primary and secondary resistance have emerged with poor outcomes and limited treatment options. The majority of chronic lymphocytic leukemia (CLL) patients who progress on covalent BTK inhibitor therapy, such as ibrutinib, become resistant to treatment due to a BTK-C481S mutation. Given the high risk of progression of disease and development of resistance to currently available BTKi's in patients with hematological malignancies, there is an unmet need for more effective and tolerable treatment. The compound of Formula I (Compound A) is an orally bioavailable, potent and reversible dual inhibitors of both wild type and C481S-mutant BTK that have demonstrated superiority over ibrutinib in CLL and DLBCL mouse models and targets ibrutinib-resistant CLL, Richter's transformation, and other B-cell malignancies (Reiff et al. Cancer Discovery. 2018, 8:1300-1315).

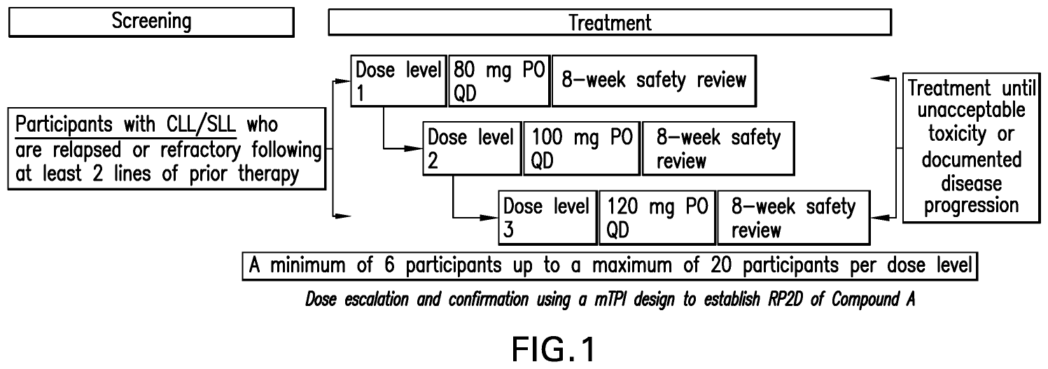

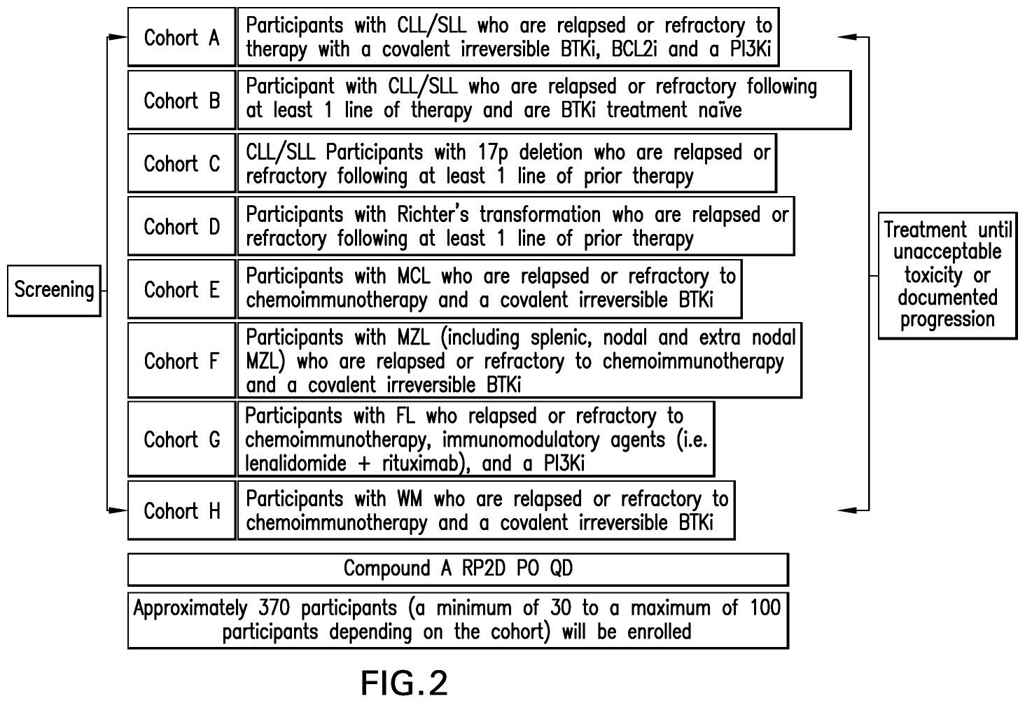

A non-randomized open-label, Phase 2 dose escalation and confirmation followed by a single-group parallel assignment study is planned in order to evaluate Compound A in approximately 400 participants. A Recommended Phase 2 Dose (RP2D) will be determined and patients with various hematological malignancies who have relapsed or are refractory to prior therapies will be evaluated. While BTK inhibitors are approved for the treatment of several lymphoproliferative malignancies, resistance is known to develop. Each hematologic malignancy that will be studied represents a significant unmet medical need. Specifically, participants with hematologic malignancies of CLL/SLL, Richter's transformation, MZL, MCL, FL, and WM will be enrolled. The first part is a dose escalation and confirmation and the second part is a cohort expansion. FIG. 1 is a scheme illustrating the dose-escalation and confirmation cohorts using a modified toxicity probability interval (mTPI) design to establish RP2D and FIG. 2 is a scheme illustrating the expansion cohort. The mTPI design has been described in Y. Ji et al., J Clin Oncol:1785-1791 (2013); Y. Ji et al., Clinical Trials, 2010, 7, 653-663; and Y. Ji et al., Clinical Trials, 2007, 4: 235-244.

The study design of the Dose-Escalation and Confirmation cohort (Part 1) and Cohort Expansion (Part 2) is summarized in Table 1 below. Male and female participants at least 18 years of age with various hematological malignancies who have either relapsed following or are refractory to available therapies will be enrolled as indicated below:

TABLE 1

| Primary Objectives | Primary Endpoints |
|---|---|
| Part 1: To determine the safety and | Dose-limiting toxicity (DLT). |
| tolerability and to establish a recommended | Adverse Event (AE). |
| Phase 2 dose (RP2D) of Compound A. | Discontinuing study intervention due to an |
| | AE. |
| Part 2: Cohorts A to C (chronic | Objective response (OR): complete response |
| Lymphocytic Leukemia [CLL]/Small | (CR), or complete response with incomplete |
| Lymphocytic Lymphoma [SLL]): To evaluate | bone marrow recovery (CRi), or nodular |
| the objective response rate (ORR) following | partial response (nPR) or partial response |
| administration with Compound A per | (PR). |
| International Workshop on CLL (iwCLL) | |
| criteria 2018 as assessed by independent | |
| central review (ICR). | |
| Part 2: Cohorts D to G (Richter's | Objective response (OR): complete response |
| Transformation [RT], Mantle cell Lymphoma | (CR) or partial response (PR). |
| [MCL], Marginal zone Lymphoma [MZL], | |
| Follicular Lymphoma [FL]): To evaluate the | |
| ORR following administration with | |
| Compound A per the Lugano criteria 2014 as | |
| assessed by ICR. | |
| Part 2: Cohort H (Waldenström's | Objective response (OR): complete response |
| Macroglobulinemia [WM]): To evaluate the | (CR), or very good partial response (VGPR) |
| ORR following administration with | or partial response (PR). |
| Compound A per International Workshop on | |
| WM (IWWM) 2014 as assessed by ICR. | |
| Secondary Objectives | Secondary Endpoints |
| Part 1: To characterize the pharmacokinetic | PK parameters including area under the |
| (PK) profile of Compound A. | curve (AUC), minimum concentration |
| | (Cmin), and maximum concentration (Cmax). |
| Part 1: To evaluate the ORR and duration of | Objective response (OR): complete response |
| response (DOR) following administration | (CR), or complete response with incomplete |
| with Compound A for CLL/SLL participants | bone marrow recovery (CRi), or nodular |
| per iwCLL criteria 2018 as assessed by ICR. | partial response (nPR) or partial response |
| | (PR). |
| | DOR, defined as the time from the first |
| | documented evidence of an objective response |
| | until disease progression or death due to any |
| | cause, whichever occurs first. |
| Part 2: All Cohorts: To determine the safety | AE. |
| and tolerability of Compound A. | Discontinuing study intervention due to an |
| | AE |
| Part 2: All Cohorts: To characterize the PK | PK parameters including AUC, Cmin, and |
| profile of Compound A. | Cmax. |
| Part 2: Cohorts A to C (CLL/SLL): To | DOR. |
| evaluate DOR following administration with | |
| Compound A per iwCLL criteria 2018 as | |
| assessed by ICR. | |
| Part 2: Cohorts D to G: (RT, MCL, MZL, | DOR. |
| FL): To evaluate the DOR following | |
| administration with Compound A per the | |
| Lugano criteria 2014 as assessed by ICR. | |
| Part 2: Cohort H (WM): To evaluate the | DOR. |
| DOR of Compound A per IWWM 2014 as | |
| assessed by ICR. | |
| Tertiary/Exploratory | |
| (Parts 1 and 2 [Cohorts B and C only]) | |
| To explore the relationship between | 12-lead electrocardiogram parameters |
| plasma concentration of Compound A and | |
| QTc Intervals | |
| (Parts 1 and 2 [Cohorts A to C only]) | |
| To evaluate response category of partial | Objective response including partial |
| response with lymphocytosis (PRL) | response with lymphocytosis (PRL), |
| following administration with Compound | defined as complete response (CR), or |
| A for CLL/SLL participants per iwCLL | complete response with incomplete bone |
| criteria 2018 as assessed by ICR. | marrow recovery (CRi), or nodular partial |
| | response (nPR) or partial response (PR) or |
| | partial response with lymphocytosis |
| | (PRL). |
| (Part 2 only) | |
| Cohorts D to G (RT, MCL, MZL, FL): To | Objective response: complete response |
| evaluate the ORR following administration | (CR) or partial response (PR). |
| with Compound A per the Cheson criteria | |
| (IWG 2007) as assessed by ICR. | |
| Cohorts H (WM): To evaluate response | Objective response including minor |
| category of minor response (MR) | response (MR), defined as complete |
| following administration with Compound | response (CR), or very good partial |
| A per IWWM 2014 as assessed by ICR. | response (VGPR), or partial response (PR) |
| | or minor response (MR). |
| Cohorts A to C (CLL/SLL): To evaluate | Minimal residual disease (MRD), defined |
| minimal residual disease (MRD) and | as having undetectable MRD (MRD-neg) |
| progression-free survival (PFS) following | remission if they have blood or marrow |
| administration with Compound A per | with less than one CLL cell per 10,000 |
| iwCLL criteria 2018 as assessed by ICR. | leukocytes (<1/10⁴). |
| | Progression-free survival (PFS), defined as |
| | the time from first dose to the first |
| | documented disease progression per |
| | disease-specific criteria as assessed by |
| | ICR, where indicated; or death due to any |
| | cause, whichever occurs first. |
| Cohorts D to G (RT, MCL, MZL, FL): To | Progression-free survival (PFS). |
| evaluate the progression-free survival | |
| (PFS) following administration with | |
| Compound A per the Lugano | |
| Classification 2014 as assessed by ICR. | |
| Cohort H (WM): To evaluate the | Progression-free survival (PFS). |
| progression-free survival (PFS) following | |
| administration with Compound A per | |
| IWWM 2014 as assessed by ICR. | |
| All cohorts: To evaluate overall survival | Overall survival (OS), defined as the time |
| (OS) following administration with | from the first dose of study treatment to |
| Compound A. | death due to any cause. |
| (Parts 1 and 2 [Cohorts A to D only]) | |
| To investigate the relationship between | BTK-C481 mutation status |
| clinical outcomes (Objective Response | |
| (OR), Duration of Response (DOR), | |
| Progression-free survival (PFS), Overall | |
| Survival (OS) of Compound A treatment | |
| and BTK-C481 mutation status | |
| All cohorts: To identify molecular | Germline genetic variation, genetic |
| (genomic, metabolic, and/or proteomic) | (deoxyribonucleic acid [DNA]) mutations |
| biomarkers that may be indicative of | from tumor, tumor and blood ribonucleic |
| clinical response/resistance, safety, | acid (RNA) variation, proteomics and |
| pharmacodynamic activity, and/or the | immunohistochemistry (IHC), and other |
| mechanism of action of Compound A. | blood-derived biomarkers. |
| All cohorts: To evaluate changes in health- | Mean score change from baseline at a pre- |
| related quality-of-life assessments from | defined timepoint evaluated by EORTC |
| baseline using the Electronic European | QLQ-C30. |
| Organization for Research and Treatment | Global Health Status/QoL (Items 29, |
| of Cancer Quality of Life Questionnaire | 30). |
| Core 30 items (eEORTC QLQ-C30). | Physical functioning (Items 1 through |
| | 5). |
| | Fatigue (Items 10, 12, 18). |
| All cohorts: To evaluate the use of health | Health Care Resource Utilization/Medical |
| services for the purpose of treating | Care Resource Utilization form. |
| participants in this study. | |
| All cohorts: To evaluate health status using | Mean change from baseline per pre- |
| the EuroQoL (EQ)-5D-5L VAS. | defined timepoint of EQ-5D-5L VAS |
| | score. |

This is a Phase 2 open-label, nonrandomized, dose escalation and confirmation followed by a single group parallel assignment study to evaluate the safety and efficacy of Compound A in approximately 400 participants with various hematological malignancies. The study will be divided into 2 parts: dose escalation and confirmation (Part 1) and cohort expansion (Part 2).

Part 1 consists of dose escalation and confirmation of the dose of Compound A in CLL/SLL participants, that aims to establish the RP2D of Compound A, which will be assessed as a primary objective. The final RP2D will be determined using PK and PD endpoints, as well as all available safety and efficacy data, including DLT rates and the cumulative incidence of late toxicities (i.e., toxicities that occur after the DLT observation period) from participants from Part 1 of this study and data from a prior study. Following determination of a RP2D, this study will proceed with Part 2 in 8 expansion cohorts (Cohorts A to H). Participants include those with various hematological malignancies.

Participants eligible for inclusion in this study must be relapsed or refractory to existing treatment.

### Dose Escalation and Confirmation (Part 1)

Approximately 30 participants, a minimum of 6 to a maximum of 20 participants per dose level with CLL/SLL will be enrolled in Part 1. Participants will include those with:
CLL/SLL who have relapsed or are refractory following at least 2 lines of prior therapy

The primary endpoints of Part 1 of the study are to evaluate the DLTs, AEs, and AEs resulting in treatment discontinuation with the aim to establish a RP2D of Compound A for Part 2. In Part 1 of the study 3 predetermined dose levels of Compound A will be evaluated:
Dose level 1 (DL1): 80 mg
Dose level 2 (DL2): 100 mg
Dose level 3 (DL3): 120 mg

A minimum of 6 participants are required for each dose level, with the potential to treat up to a maximum of 20 participants depending on dose decisions. A minimum of 8 weeks of safety data will be reviewed for the first 10 participants treated at a dose level before escalation decisions are made. Dose escalation and de-escalation rules are based on the mTPI design. The decision to escalate will be based on safety; however, the totality of the available data, including PK, PD, and efficacy data will be taken into consideration. After DLT evaluation period, the participants will continue to be followed for a total of 12 weeks for safety and efficacy evaluation. Thereafter, DLTs will be monitored every 6 months to evaluate accumulated safety data. The totality of the data will be used to determine the final RP2D for Part 2 (cohort expansion).

### Cohort Expansion (Part 2)

Approximately 370 participants will be enrolled in Part 2: 100 participants for Cohort A, 30 participants in each of the following Cohorts B, C, D, and F, and 50 participants in each of the following Cohorts E, G, and H. Participants will be enrolled within disease-specific cohorts as follows:
A. CLL/SLL who are relapsed or refractory to prior therapy with a covalent, irreversible BTKi, a BCL2i, and a PI3Ki.
B. CLL/SLL who are relapsed or refractory following at least 1 line of prior therapy and are BTKi treatment naïve.
C. CLL/SLL with 17p deletion who are relapsed or refractory following at least 1 line of prior therapy.
   Note: Participants with the 17p deletion will be assigned to Cohort C preferentially.
D. Richter's transformation who are relapsed or refractory following at least 1 line of prior therapy.
E. MCL who are relapsed or refractory to chemoimmunotherapy and a covalent irreversible BTKi.
F. MZL (including splenic, nodal, and extra nodal MZL) who are relapsed or refractory to chemoimmunotherapy and a covalent irreversible BTKi.
G. FL who are relapsed or refractory to chemoimmunotherapy, immunomodulatory agents (i.e. lenalidomide+rituximab), and a PI3Ki.
H. WM who are relapsed or refractory to chemoimmunotherapy and a covalent irreversible BTKi.

All cohorts will receive Compound A monotherapy.

There will be one efficacy interim analysis per cohort planned for futility checking. An interim safety analysis for DLT assessment of Part 2 participants will be performed every 6 months since first participant is enrolled.

After signing the informed consent, suitable candidates will be screened to assess whether they meet all study eligibility criteria. Eligible participants will be assigned to 1 of 8 cohorts (Cohorts A to H), according to their hematologic malignancy and prior therapy received. The study will be conducted in conformance with Good Clinical Practice (GCP).

The primary endpoint of Part 2 of the study is Objective Response (OR), defined as at least partial response (PR), assessed by specific response criteria for each hematologic malignancy. Secondary endpoints include safety, PK, and duration of response (DOR).

For both Parts, treatment with Compound A monotherapy will continue until unacceptable toxicity, documented progression, or another discontinuation criterion is met.

AEs will be monitored throughout the study and graded in severity according to the guidelines outlined in the NCI CTCAE version 5.0. Each participant will be monitored for AEs and severe adverse events (SAEs) for 30 day and 90 days, respectively, after discontinuation of study intervention.

### Scientific Rationale for Study Design

This is a multicenter, non-randomized open-label, Phase 2 dose escalation and confirmation followed by a multi-cohort single-group study. This study is designed to first establish the Recommended Phase 2 Dose (RP2D) and then as a proof-of-concept to assess the efficacy and safety of Compound A monotherapy across cohorts of participants with various hematological malignancies who have relapsed or are refractory to prior therapies. BTK inhibitors are approved for the treatment of several lymphoproliferative malignancies; however, resistance is known to develop. These hematologic malignancies were selected because each represents a significant unmet medical need and prior BTK inhibitors have shown efficacy in these malignancies.

### Rationale for Endpoints

### Objective Response Rate

Objective Response Rate (ORR) is the primary efficacy endpoint for Part 2 of the study. ORR is a secondary endpoint for Part 1 of the study.

Treatment effect measured by ORR can represent direct clinical benefit based on the specific disease, context of use, magnitude of the effect, number of CRs, durability of response, disease setting, location of the tumors, available therapy, and risk-benefit relationship. Treatment effect measured by ORR can be a surrogate endpoint to support accelerated approval according to FDA guidance (Clinical Trial Endpoints for the Approval of Cancer Drugs and Biologics Guidance for Industry, December 2018). Nodular partial remission (nPR) has all the features of a CR but has lymphoid nodules in the marrow which should be further evaluated. If immunohistochemistry conclusively shows the nodules are CLL cells, a response of “nPR” should be assigned. If analysis shows them to be composed of other cell types, it should be considered CR.

Each hematological malignancy has specific response criteria developed by experts that will be applied in the assessment of ORR (for Cohorts A to H).

### Duration of Response

Duration of Response (DOR) is a secondary efficacy endpoint for Part 1 and Part 2 of the study. Improved DOR can result in a meaningful delay in disease progression as opposed to a temporary response without lasting benefit.

### Exploratory Efficacy Endpoints

Exploratory efficacy objectives for Part 1 of this study include evaluation of PRL in CLL/SLL participants. Exploratory efficacy objectives for Part 2 of this study include evaluation of PRL (Cohorts A to C only), ORR (Cohorts D to G only), MR (Cohort H only), MRD (Cohorts A to C only), PFS, and OS of Compound A in each of the cohorts. OS represents a precise and reliable measure of time to event endpoint. PFS is a surrogate endpoint that reflects tumor growth and includes deaths and therefore correlates to OS. MRD is a surrogate endpoint for CLL and correlates with improved OS and PFS. PRL represents a reduction in lymph nodes, splenomegaly and other markers of response with no sign of progression other than lymphocytosis, and has been used in other BTKi studies. Partial response with lymphocytosis is defined as a >50% reduction in lymphadenopathy and splenomegaly, with persistent lymphocytosis. Objective Response Rate including MR is defined as the percentage of participants who achieve CR, VGPR, PR, or MR.

### Safety Endpoints

The safety and tolerability of Compound A will be assessed by clinical evaluation of AEs and inspection of other study parameters including vital signs, physical examination, and laboratory safety tests at time points specified in the SoA. AEs will be graded and recorded. In addition, DLTs will be used to determine the RP2D of Compound A.

### Patient-Reported Outcomes

In support of the exploratory objective in Part 1 and 2 of this study to evaluate changes in patient-reported outcomes from baseline, the EORTC QLQ-C30 and EQ-5D-5L questionnaires will be used. These patient-reported assessments are not pure efficacy or safety endpoints because they are affected by both disease progression and treatment tolerability.

### EORTC QLQ-C30

EORTC QLQ-C30 is the most widely used cancer-specific, health-related, QoL instrument. It contains 30 items and measures 5 functional dimensions (physical, role, emotional, cognitive, and social), 3 symptom items (fatigue, nausea/vomiting, and pain), and 6 single items (dyspnea, sleep disturbance, appetite loss, constipation, diarrhea, and financial impact). It is scored on a 4-point scale (1=not at all, 2=a little, 3=quite a bit, 4=very much). The EORTC QLQ-C30 instrument also contains 2 global scales that use 7-point scale scoring with anchors (1=very poor and 7=excellent). The EORTC QLQ-C30 is a psychometrically and clinically validated instrument appropriate for assessing QoL in oncology studies. It has been translated and validated into over 100 languages and is used in more than 3,000 studies worldwide.

### EUROQOL EQ-5D

The EQ-5D-5L is a standardized instrument for use as a measure of health outcome and will provide data to develop health utilities for use in health economic analyses. The health state dimensions in the EQ-5D-5L include the following: mobility, self-care, usual activities, pain/discomfort, and anxiety/depression. Each dimension is rated on a 5-point scale from 1 (no problem) to 5 (unable to/extreme problems). The EQ-5D-5L also includes a graded (0 to 100) vertical visual analog scale on which the participant rates his or her general state of health at the time of the assessment. This instrument has been used extensively in cancer studies and published results from these studies support its validity and reliability.

### Pharmacokinetic Endpoints

In support of the secondary objective to characterize the PK profile of Compound A monotherapy in Parts 1 and 2 of the study, the PK endpoints will include AUC, Cₘᵢₙ, and Cₘₐₓ. Compound A concentrations will serve as the primary readout for PK, and these data will be used to derive PK parameters of Compound A. The results of these analyses will be used in conjunction with the efficacy, safety and pharmacodynamics data to help assess dosing of Compound A.

### Pharmacodynamic Endpoints

In support of the exploratory objective in Parts 1 and 2 of this study to identify molecular biomarkers that may be indicative of clinical response/resistance, safety, pharmacodynamic activity, and/or the mechanism of action of Compound A, germline genetic variation, genetic variation, proteomics and IHC, and other blood-derived biomarkers will be evaluated. The relationship between plasma concentration of Compound A and QTc Intervals will be explored by reviewing ECG parameters.

### Overall Design:

| | |
|---|---|
| Study Phase | Phase 2 |
| Primary Purpose | Treatment |
| Indication | Treatment of participants with hematologic malignancies |
| Population | Participants with CLL, nonHodgkin Lymphoma, and WM |
| Study Type | Interventional |
| Intervention Model | Parallel |
| | This is a multi-site study |
| Type of Control | No treatment control |
| Study Blinding | Unblinded Open-label |
| Blinding Roles | No Blinding |
| Estimated Duration | The Sponsor estimates that the study will require approximately 78 |
| of Study | months from the time the first participant signs the informed |
| | consent until the last participant's last study-related telephone call |
| | or visit. |

### Number of Participants

Participants with various hematological malignancies will be treated in the dose escalation and confirmation (Part 1) and cohort expansion (Part 2) of the study. In Part 1 approximately 30 participants, a minimum of 6 participants with the potential to treat up to a maximum of 20 participants depending on dose decisions, will be enrolled at each dose level. In Part 2, approximately 370 participants (a target of 100 total participants for Cohort A; 50 total participants in each of the following Cohorts E, G, and H; and 30 total participants in each of the following Cohorts B, C, D, and F) will be enrolled.

1. A method of treating a BTK mediated disorder, comprising administering to a subject in need thereof a compound of Formula (I): 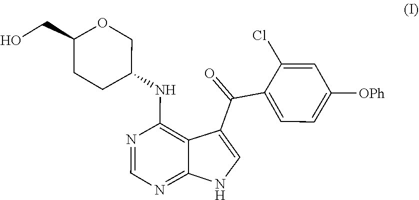 [SMILES: O=C(c1ccc(Oc2ccccc2)cc1Cl)c1c[nH]c2ncnc(N[C@@H]3CC[C@@H](CO)OC3)c12]
or a pharmaceutically acceptable salt thereof, in an amount of about 80 to about 160 mg per day.

2. The method of claim 1, wherein the compound is administered in an amount from about 80 mg to about 120 mg per day.

3. The method of claim 1, wherein the compound is administered in an amount of about 80 mg per day.

4. The method of claim 1, wherein the compound is administered in an amount of about 100 mg per day.

5. The method of claim 1, wherein the compound is administered one, two, three, or four times per day.

6. The method of claim 5, wherein the compound is administered once per day.

7. The method of of claim 1, wherein the compound is administered every day for at least one, two, three, four, five, six, seven, eight, nine, or ten days.

8. The method of of claim 1, wherein the compound is administered every day for at least one, two, three, four, five, six, seven, eight, nine, or ten weeks.

9. The method of of claim 1, wherein the compound is administered for at least one day per week, at least two days per week, at least three days per week, at least four days per week, at least five days per week, or at least six days per week.

10. The method of of claim 1, wherein the compound is administered for a period of at least one week, two weeks, three weeks, four weeks, five weeks, six weeks, seven weeks, eight weeks, nine weeks, or ten weeks.

11. The method of of claim 1, wherein the compound is administered for a period of at least three months, four months, five months, six months, seven months, eight months, nine months, ten months, eleven months, twelve months, eighteen months, twenty-four months, three years, four years, or five years.

12. The method of of claim 1, wherein the BTK-mediated disorder is cancer.

13. The method of claim 12, wherein the cancer is a hematological malignancy selected from chronic lymphocytic leukemia (CLL), Richter's Transformation (RT), small lymphocytic lymphoma (SLL), diffuse large B-cell lymphoma (DLBCL), follicular lymphoma (FL), mantle cell lymphoma (MCL), primary central nervous system (CNS) lymphoma, secondary central nervous system (CNS) lymphoma, marginal zone lymphoma (MZL), and Waldenström's macroglobulinemia (WM).

14. The method of claim 13, wherein the hematological malignancy is selected from Chronic Lymphocytic Leukemia (CLL), Small Lymphocytic Leukemia (SLL), Richter's Transformation (RT), Mantle cell Lymphoma (MCL), Marginal zone Lymphoma (MZL), Follicular Lymphoma (FL), and Waldenström's Macroglobulinemia (WM).

15. The method of claim 12 wherein the cancer or hematological malignancy is relapsed or refractory to at least one prior therapy.

16. The method of claim 12, wherein the cancer or hematological malignancy is ibrutinib resistant.

17. A method of treating cancer, comprising administering to a subject in need thereof a compound of Formula (I): 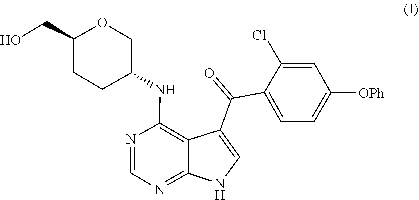 [SMILES: O=C(c1ccc(Oc2ccccc2)cc1Cl)c1c[nH]c2ncnc(N[C@@H]3CC[C@@H](CO)OC3)c12]
or a pharmaceutically acceptable salt thereof, in an amount of about 80 mg per day, wherein the cancer is a hematological malignancy selected from chronic lymphocytic leukemia (CLL), Richter's Transformation (RT), small lymphocytic lymphoma (SLL), diffuse large B-cell lymphoma (DLBCL), follicular lymphoma (FL), mantle cell lymphoma (MCL), primary central nervous system (CNS) lymphoma, secondary central nervous system (CNS) lymphoma, marginal zone lymphoma (MZL), Waldenström's macroglobulinemia (WM), acute myeloid leukemia (AML), multiple myeloma (MM), and pediatric sarcoma and pediatric brain tumors.

18. The method of claim 17, wherein the hematological malignancy is chronic lymphocytic leukemia (CLL) or small lymphocytic lymphoma (SLL).

19. The method of claim 17, wherein the hematological malignancy is Richter's Transformation (RT).

20. The method of claim 17, wherein the hematological malignancy is follicular lymphoma (FL).

21. The method of claim 17, wherein the hematological malignancy is marginal zone lymphoma (MZL).

22. The method of claim 17, wherein the hematological malignancy is mantle cell lymphoma (MCL).

23. The method of claim 17, wherein the hematological malignancy is Waldenström's macroglobulinemia (WM).
